SEQUENCE LISTING (13 sequences)

SEQ ID NO: 1 - protein, 349 aa, Artificial Sequence
  Human NTCP Protein
MEAHNASAPFNFTLPPNFGKRPTDLALSVILVFMLFFIMLSLGCTMEFSKIKAHLWKPKGLAIALVAQYGIMPLTAFVLGKVFRLKNIEALAILVCGCSPGGNLSNVFSLAMKGDMNLSIVMTTCSTFCALGMMPLLLYIYSRGIYDGDLKDKVPYKGIVISLVLVLIPCTIGIVLKSKRPQYMRYVIKGGMIIILLCSVAVTVLSAINVGKSIMFAMTPLLIATSSLMPFIGFLLGYVLSALFCLNGRCRRTVSMETGCQNVQLCSTILNVAFPPEVIGPLFFFPLLYMIFQLGEGLLLIAIFWCYEKFKTPKDKTKMIYTAATTEETIPGALGNGTYKGEDCSPCTA

SEQ ID NO: 2 - protein, 9 aa, Artificial Sequence
  Human NTCP Protein Fragment
KGIVISLVL

SEQ ID NO: 3 - protein, 47 aa, Artificial Sequence
  Hepatitis B Virus Pre S1 domain
GTNLSVPNPLGFFPDHQLDPAFGANSNNPDWDFNPNKDHWPDANKVG

SEQ ID NO: 4 - protein, 45 aa, Artificial Sequence
  Hepatitis CD8 hinge spacer
TTTPAPRPPTPAPTIASQPLSLRPEACRPAAGGAVHTRGLDFACD

SEQ ID NO: 5 - protein, 24 aa, Artificial Sequence
  Hepatitis CD8 Transmembrane domain
IYIWAPLAGTCGVLLLSLVITLYC

SEQ ID NO: 6 - protein, 41 aa, Artificial Sequence
  Human CD28 constimulatory domain
RSKRSRLLHSDYMNMTPRRPGPTRKHYQPYAPPRDFAAYRS

SEQ ID NO: 7 - protein, 42 aa, Artificial Sequence
  Human 4-1BB contiumlatory domain
KRGRKKLLYIFKQPFMRPVQTTQEEDGCSCRFPEEEEGGCEL

SEQ ID NO: 8 - protein, 112 aa, Artificial Sequence
  Human CD3 zeta costimulatory domain
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 18 aa, Artificial Sequence
  Artificial cleavage sequence T2A
EGRGSLLTCGDVEENPGP

SEQ ID NO: 10 - protein, 239 aa, Artificial Sequence
  Artificial EGFP marker domain
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 11 - protein, 589 aa, Artificial Sequence
  Artificial CAR construct
MALPVTALLLPLALLLHAARPGTNLSVPNPLGFFPDHQLDPAFGANSNNPDWDFNPNKDHWPDANKVGTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 12 - DNA, 1770 bp, Artificial Sequence
  Artificial CAR Sequence
atggccttaccagtgaccgccttgctcctgccgctggccttgctgctccacgccgccaggccgggcacaaatctgagcgtgccaaatcctctgggattcttcccagaccaccagctggatcctgccttcggcgccaacagcaacaaccccgactgggacttcaaccccaacaaggaccattggcctgacgccaacaaagtgggcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatttgggcccctctggctggtacttgcggggtcctgctgctttcactcgtgatcactctttactgtaggagcaagcggagcagactgctgcacagcgactacatgaacatgaccccccggaggcctggccccacccggaagcactaccagccctacgcccctcccagggatttcgccgcctaccggagcaagcgcggtcggaagaagctgctgtacatctttaagcaacccttcatgaggcctgtgcagactactcaagaggaggacggctgttcatgccggttcccagaggaggaggaaggcggctgcgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgagggcaggggaagtcttctaacatgcggggacgtggaggaaaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 13 - protein, 21 aa, Artificial Sequence
  Human CD8 signal peptide
MALPVTALLLPLALLLHAARP

(19) United States
(12) Patent Application Publication
(10) Pub. No.: US 2022/0152108 A1
(43) Pub. Date: May 19, 2022

(54) CHIMERIC ANTIGEN RECEPTORS DIRECTED TO CELLS EXPRESSING THE SODIUM TAUROCHOLATE CO-TRANSPORTING RECEPTOR

(71) Applicant: Svenska Vaccinfabriken Produktion AB, Stockholm (SE)

(72) Inventors: Matti Sällberg, Stockholm (SE); Lars Frelin, Stockholm (SE); Margaret Sällberg-Chen, Stockholm (SE); Anna Pasetto, Stockholm (SE)

(21) Appl. No.: 17/440,135

(22) PCT Filed: Mar. 17, 2020
(86) PCT No.: PCT/US2020/023137

§ 371 Date: Sep. 16, 2021

Related U.S. Application Data
(60) Provisional application No. 62/820,024, filed on Mar. 18, 2019.

(51) Int. Cl. A61K 35/17; C07K 14/725

(52) U.S. Cl. CPC A61K 35/17; C07K 14/7051

(57) ABSTRACT

Aspects of the invention described herein relate to a chimeric antigen receptor (CAR), which can be presented on a cell or vesicle, which comprises the preS1 domain of the hepatitis B virus envelope protein and, which binds to cells expressing the sodium taurocholate co-transporting receptor (NTCP), such as NTCP expressing liver cells. Compositions comprising said CARs including cells and vesicles, methods of making said compositions, and methods of using said compositions to deliver a nucleic acid, protein, peptide, or drug, such as CRISPR/CAS system so as to treat, inhibit, or ameliorate a liver disease, such as liver cancer or pathogens infecting liver cells or to modulate a function of the liver cells are contemplated.

### RELATED APPLICATION

This application claims priority to U.S. Provisional Patent Application No. 62/820,024, filed Mar. 18, 2019, the entire content of which is expressly incorporated by reference in its entirety.

### FIELD OF THE INVENTION

Some embodiments of the methods and compositions provided herein relate to chimeric antigen receptor (CAR) T cells, which comprise the preS1 domain of the hepatitis B virus envelope protein and, which bind to cells expressing the sodium taurocholate co-transporting receptor (NTCP), such as NTCP expressing liver cells. Compositions comprising said CAR expressing cells or vesicles, methods of making said CAR expressing cells or vesicles, and methods of using said CAR expressing cells or vesicles to treat, inhibit, or ameliorate NTCP expressing cells or a liver disease, such as liver cancer or pathogens infecting liver cells are contemplated.

### BACKGROUND OF THE INVENTION

The adoptive transfer of human T lymphocytes that are engineered by gene transfer to express chimeric antigen receptors (CARS) specific for surface molecules expressed on cells has the potential to effectively treat a wide-range of diseases. Chimeric receptors are synthetic receptors that include an extracellular ligand binding domain, most commonly a single chain variable fragment of a monoclonal antibody (scFv) linked to intracellular signaling components, most commonly CD3ζ alone or combined with one or more costimulatory domains. Much of the research in the design of chimeric antigen receptors has focused on defining scFvs and other ligand binding elements that target cells without causing serious toxicity to essential normal tissues, and on defining the optimal composition of intracellular signaling modules to activate T cell effector functions. There remains a need for a CAR T cell-mediated therapy that is selective for specific targets and, which minimizes adverse side effects.

### SUMMARY OF THE INVENTION

Some embodiments provided herein include compositions, such as a cells, preferably T cells, comprising one or more CARs, which comprise the preS1 domain of the hepatitis B virus envelope protein, and nucleic acids and polypeptides encoding said CARS. Additional embodiments include methods of making and using said cells, preferably T cells, comprising said CARs to interact with or to bind to NTCP expressing cells, such as liver cells, and methods of modulating the interaction or binding of said cells comprising said one or more CARs, preferably T cells, to said NTCP expressing cells by administering or providing an antibody or binding fragment thereof, such as an scFv or Fab fragment, or peptide, which binds to or interacts with said preS1 domain of said CARs. In some embodiments, cells, preferably T cells, comprising one or more CARs, which comprise the preS1 domain of the hepatitis B virus envelope protein, are administered to a subject having NTCP expressing cells, preferably a human and, optionally, subsequent to said administration, an antibody or binding fragment thereof, such as an scFv or Fab fragment, or peptide, which binds to or interacts with said preS1 domain of said CARs is administered to said subject.

Preferred alternatives include the following.

1. A composition comprising a nucleic acid encoding SEQ ID NO: 12, such as a cell or vesicle.

2. The composition of alternative 1, wherein said composition is a plasmid or vector, such as a lentiviral vector or a cell, such as a T cell or a vesicle, such as an exosome.

3. A composition comprising a polypeptide encoding SEQ ID NO: 11, such as a cell or vesicle.

4. The composition of alternative 3, wherein said composition is a cell, such as a T cell.

5. The composition of any one of alternatives 1-4, wherein said composition is a white blood cells, an NK cell or a CD8+ T cytotoxic lymphocyte cell selected from the group consisting of naïve CD8+ T cells, central memory CD8+ T cells, effector memory CD8+ cells and bulk CD8+ T cells or a CD4+ T helper lymphocyte cell that is selected from the group consisting of naïve CD4+ T cells, central memory CD4+ T cells, effector memory CD4+ T cells, and bulk CD4+ T cells.

6. A composition, such as a cell, e.g., a White blood cell or T cell, or a vesicle, e.g., an exosome, comprising a polypeptide that comprises a PreS1 domain amino acid sequence set forth by SEQ ID NO: 3, optionally SEQ. ID. NO: 13, further comprising any one or more of: a CD8 hinge spacer set forth by SEQ ID NO: 4, a CD8 Transmembrane domain set forth by SEQ ID NO: 5, a CD28 domain set forth by SEQ ID NO: 6, a 4-1BB domain set forth by SEQ ID NO: 7, a CD3 zeta domain set forth by SEQ ID NO: 8, or a EGFP domain set forth by SEQ ID NO: 10 or a nucleic acid encoding said polypeptide sequence or a nucleic acid encoding said polypeptide.

7. A composition, such as a cell, e.g., a white blood cell or T cell, or a vesicle, e.g., an exosome, comprising a polypeptide that comprises at least 90%, 91%, 92%, 93%, 94%, 95%, 96%, 97%, 98%, 99%, or 100% sequence identity to a PreS1 domain amino acid sequence set forth by SEQ ID NO: 3, optionally se. ID. NO: 13, further comprising any one or more of: a CD8 hinge spacer having at least 90%, 91°/i, 92% 93%, 94%, 95%, 96%, 97%, 98%, 99%, or 100% sequence identity to SEQ ID NO: 4, a CD8 Transmembrane domain having at least 90%, 91%, 92%, 93%, 94%, 95%, 96%, 97%, 98%, 99%, or 100% sequence identity to SEQ ID NO: 5, a CD28 domain having at least 90%, 91%, 92%, 93%, 94%, 95%, 96%, 97%, 98%, 99%, or 100% sequence identity to SEQ ID NO: 6, a 4-1BB domain having at least 90%, 91%, 92%, 93%, 94%, 95%, 96%, 97%, 98%, 99%, or 100% sequence identity to SEQ ID NO: 7, a CD3 zeta domain having at least 90%, 91%, 92%, 93%, 94%, 95%, 96%, 97%, 98%, 99%, or 100% sequence identity to SEQ ID NO: 8, or a EGFP domain having at least 90%, 91%, 92%, 93%, 94%, 95%, 96%, 97%, 98%, 99%, or 100% sequence identity to SEQ ID NO: 10 or a nucleic acid encoding said polypeptide.

8. The composition of any one of alternatives 6 or 7, wherein said composition is a T cell, such as a CD8+ T cytotoxic lymphocyte cell selected from the group consisting of naïve CD8+ T cells, central memory CD8+ T cells, effector memory CD8+ cells and bulk CD8+ T cells or a CD4+ T helper lymphocyte cell that is selected from the group consisting of naïve CD4+ T cells, central memory CD4+ T cells, effector memory CD4+ T cells, and bulk CD4+ T cells.

9. An in vivo or in vitro method of directing a cell comprising a CAR to a cell having a sodium taurocholate co-transporting receptor (NTCP) comprising:

contacting the cell comprising the NTCP with any one or more of the cells of any one or more of alternatives 1-8 in vivo or in vitro.

10. An in vivo or in vitro method of inhibiting a pathogen, such as a viral pathogen, or a cancer, such as hepatocellular carcinoma, which is present in a cell that expresses a sodium taurocholate co-transporting receptor (NTCP) comprising:

contacting the cell comprising the NTCP with any one or more of the cells of any one or more of alternatives 1-8 thereby inhibiting the pathogen or cancer in vivo or in vitro.

11. An in vivo or in vitro method of inducing a signal or modulating a function in a cell that expresses a sodium taurocholate co-transporting receptor (NTCP) comprising:

contacting the cell comprising the NTCP with any one or more of the cells of any one or more of alternatives 1-8 in vivo or in vitro.

12. A method of treating, inhibiting, or ameliorating a pathogenic infection, such as a viral infection, or a cancer, such as a liver cancer or hepatocellular carcinoma in a subject, preferably a human, comprising administering the any one or more of the cells of any one or more of alternatives 1-8 to said subject, optionally selecting said subject to receive said administration, e.g., by diagnostic or clinical evaluation and, optionally measuring an inhibition, amelioration, or treatment of said pathogenic infection or cancer in said subject or a marker thereof.

13. The method of any one of alternatives 9-12, further comprising contacting the cells of any one of alternatives 1-8 with monoclonal or polyclonal antibodies or binding fragments thereof, such as scFv or Fab fragments, preferably humanized, wherein said monoclonal or polyclonal antibodies or binding fragments thereof bind to epitopes present on a preS1 domain of the hepatitis B virus envelope protein, such as mAb 2D3, anti-preS1 antibody available under catalog number ABIN934315, Recombinant Humanized Anti-HBV preS1 Antibody (HzKR127), Recombinant Anti-HBV preS1 Antibody (MOB-437), Recombinant Humanized Anti-HBV preS1 Antibody scFv Fragment (HzKR127)(PSBC-054), Recombinant Anti-HBV preS1 Antibody scFv Fragment (MOB-437-S(P)), Recombinant Anti-HBV preS1 Antibody scFv Fragment (MOB-799-S(P)), Recombinant Human Anti-HBV preS1 Antibody Fab Fragment (2H5-A14) (PFBC-406), Recombinant Human Anti-HBV preS1 Antibody Fab Fragment (MHH-799-F(E)), Recombinant Human Anti-HBV preS1 Antibody scFv Fragment (MHH-799-S(P)), or Recombinant Anti-HBV preS1 Antibody Fab Fragment (MOB-437-F(E)) or binding fragments thereof.

14. The method of any one of alternatives 9-12, further comprising contacting the cells of any one of alternatives 1-8 with a peptide, which binds to or interacts with a preS1 domain, such as full-length NTCP protein, e.g., SEQ ID NO 1, which can be modified e.g., by pegylation or conjugation to an albumin or a variant or mutant or derivative thereof, or a preS1 domain binding fragment of NTCP, such as a peptide comprising residues 157-165 of a human NTCP protein sequence, which can comprise the amino acid sequence of SEQ NO. 2 and, which can be modified e.g., by pegylation or conjugation to an albumin or variant, or mutant or derivative thereof.

15. Use of the cells of any one of alternatives 1-8 as a medicament. 16. The cells of anyone of alternatives 1-8 for use in treating, inhibiting, or ameliorating a pathogenic infection of a liver of a subject or for use in treating, inhibiting, or ameliorating a liver disease, such as a liver cancer of a subject e.g., hepatocellular carcinoma or to modulate a function of a liver cell.

17. The use of alternative 15 or the cells of alternative 16 further comprising using the antibody set forth in alternative 13 or the binding fragment thereof or the peptide of alternative 14 or the binding fragment thereof or the derivative, variant, or mutant thereof to inhibit, reduce, or modulate the binding of the cells having a preS1-containing CAR to the cells expressing the NTCP e.g., to inhibit or reduce an adverse event, such as a cytokine storm.

18. The composition of any one or more of alternatives 1-8 or the cells or vesicles of alternative 16, wherein said cells or vesicles further comprise or encode a CRISPR/CAS system.

19. The composition of any one or more of alternatives 1-8 or the cells or vesicles of alternative 16, wherein said cells or vesicles further comprise a nucleic acid, protein, peptide, or drug for delivery to an NTCP-expressing cell, such as a liver cell.

20. The method of any one or more of alternatives 9-14 or the uses of alternatives 15 or 17, wherein said cells or vesicles further comprise or encode a CRISPR/CAS system.

21. The method of any one or more of alternatives 9-14 or the uses of alternatives 15 or 17, wherein said cells or vesicles further comprise a nucleic acid, protein, peptide, or drug for delivery to an NTCP-expressing cell, such as a liver cell.

### BRIEF DESCRIPTION OF THE DRAWINGS

FIG. 1 depicts a diagram showing a full length CAR as set forth herein. A 47 amino acid preS1 sequence from hepatitis B virus envelope protein was synthesized and sub-cloned into a self-inactivating lentivirus vector. The insert was confirmed by Sanger sequencing.

FIG. 2 shows pVax.10 TCR expression in total cell population 18 hours after electroporation, wherein the cells were gated on lymphocytes, single cells, live cells and CD3+ cells. Transfection with CAR-PreS1 mRNA induces GFP expression in 30% of CD3+ T cells.

FIG. 3 shows the expression of CD107 by T cells, which were co-cultured with HepG2 cells or HepG2 cells expressing or lacking NTCP receptor to which PreS1 binds.

FIG. 4 shows the expression of IFN gamma by T cells, which were co-cultured with HepG2 cells or HepG2 cells expressing or lacking NTCP receptor to which PreS1 binds

FIG. 5 shows the expression of TNT alpha by T cells, which were co-cultured with HepG2 cells or HepG2 cells expressing or lacking NTCP receptor to which PreS1 binds

FIG. 6 shows that the presence of NTCP on the hepatocytes induced an 2-fold increase in degranulation and a 3-fold increase in IFN gamma expression.

### DETAILED DESCRIPTION

The preS1 domain of the hepatitis B virus envelope protein is responsible for targeting HBV to hepatocytes expressing the sodium taurocholate co-transporting receptor (NTCP). This targeting can be used to deliver cells having preS1-containing CARs, for example immune cells, such as T cells, to the liver where they can selectively kill, inhibit or modify the liver cells, or pathogens or processes inside the liver cells. It is envisioned that the preS1 sequence can be linked to CARs so as to target said CAR expressing cells or vesicles cells e.g., immune cells, such as T cells, to liver cells, which express NTCP. When a cell or vesicle expressing the preS1 molecule on its surface, e.g., a T cell, encounters an NTCP expressing liver cell then the cells having the preS1-containing CAR will become activated through a signal transduction pathway that then triggers a signal in the cell to kill, inhibit or functionally modify the NTCP expressing liver cell. By administering the cells having the preS1 domain-containing CARs to subjects, preferably humans, having a liver disease, such as a liver cancer, the cells comprising the preS1 domain-containing CARs will efficiently eliminate the cells expressing NTCP, and thereby treat, inhibit, or ameliorate, the liver cancer, such as hepatocellular carcinoma and pathogens, such as viral pathogens, infecting the NTCP-expressing liver cells, or otherwise functionally modify the NTCP-expressing liver cells. Similarly, one can use CAR expressing cells or vesicles, which have the preS1 domain or a fragment thereof on its surface to deliver a molecule to NTCP expressing cells, such as liver cells and such molecules can comprise a nucleic acid, such as a DNA, RNA, siRNA, or miRNA, or a peptide or protein, or both or a drug or toxin.

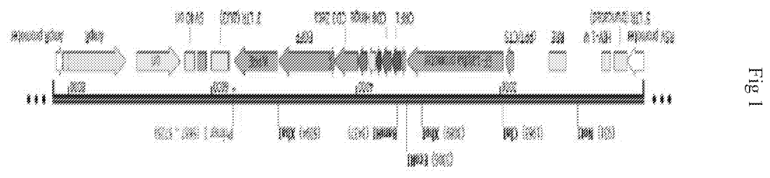

A number of preS1 domain sequence variants can be joined at different positions with a surface expressed cellular receptor, such as a CAR, at a position that is accessible to the NTCP domain. By one approach, a 47 amino acid preS1 domain sequence from hepatitis B virus envelope protein was synthesized and sub-cloned into a self-inactivating lentivirus vector. The insert was confirmed by Sanger sequencing (see FIG. 1). The construct is then tested for recognition and killing of NTCP expressing hepatoma cells, such as for example Huh-7, Huh-7.5, HepG2, or HepRG. The constructs can also be tested for recognition of these cells without killing but with the ability to inhibit HBV replication HCVcc replication, or HCV replicon replication. By this approach, constructs are selected for the ability to perform different functions depending on the signal that is transduced. It is very beneficial that these constructs are independent of the HLA set of the host and specifically target only cells expressing NTCP.

In some embodiments, the cells or vesicles, which comprise a CAR comprising the preS1 domain of the hepatitis B virus envelope protein further comprise one or more nucleic acids, which can be delivered to the NTCP expressing cells, such as liver cells. As used herein, “nucleic acid”, “nucleic acid molecule” or “polynucleotide” refer to molecules such as deoxyribonucleic acid (DNA) or ribonucleic acid (RNA), oligonucleotides, fragments generated by the polymerase chain reaction (PCR), and fragments generated by any of chemical synthesis, in vitro transcription, ligation, scission, endonuclease action, or exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and/or carbocyclic sugar analogs. Examples of modifications in a base moiety include alkylated purines, pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded.

As used herein, “coding strand” has its plain and ordinary meaning when read in light of the specification, and may include but is not limited to, for example, the DNA strand which has the same base sequence as the RNA transcript produced (although with thymine replaced by uracil). It is this strand, which contains codons, while the non-coding strand contains anti-codons.

As used herein, “coding for” or “encoding” are used herein, and refer to the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

As used herein, “regulatory elements” have their plain and ordinary meaning when read in light of the specification, and may include but is not limited to, for example, a segment of a nucleic acid molecule, which is capable of increasing or decreasing the expression of specific genes within an organism, e.g., one that has the ability to affect the transcription and/or translation of an operably linked transcribable DNA molecule. Regulatory elements such as promoters, leaders, introns, or transcription termination regions are DNA molecules that have gene regulatory activity and play an integral part in the overall expression of genes in living cells. Without being limiting, examples of regulatory elements can include, CAAT box, CCAAT box, Pribnow box, TATA box, SECIS element, mRNA Polyadenylation signals, A-box, Z-box, C-box, E-box, G-box, hormone responsive elements, such as insulin gene regulatory sequences, DNA binding domains, activation domains, or enhancer domains.

Some embodiments provided herein include cell or vesicle having a CAR, which comprises the preS1 domain of the hepatitis B virus envelope protein and a delivery payload, which can comprise a clustered regularly interspaced short palindromic repeats (CRISPR)/CAS system. A “CRISPR/CAS system” as described herein refers to a gene editing system in which expression of one or two component guide RNAs that are complimentary to a gene that is to be knocked out or expressed is necessary for a CRISPR/CAS system to function. Cas9 is an RNA-guided DNA endonuclease enzyme that associates with a one or two component guide RNA, allowing the complex to cleave a sequence that is complementary to the targeting portion of the guide RNA. In some embodiments, the basic components of CRISPR/CAS system comprise a target gene, a guide RNA, and a Cas9 endonuclease, derivative, or fragment thereof. In some embodiments described herein, cells or vesicles having a CAR with said preS1 domain also comprise a CRISPR/Cas9 system for gene editing, which will deliver the guide RNAs efficiently to NTCP expressing cell types, such as liver cells. In some embodiments, delivery can include in vitro generated guide RNA as a nucleic acid, such as where the guide RNA generated by in vitro transcription or chemical synthesis. In some embodiments the nucleic acid encoding the guide RNA is rendered nuclease resistant by incorporation of modified bases, such as 2′O-methyl bases.

Some embodiments provided herein include a CRISPR/Cpf1 system incorporated into a cell or vesicle comprising a CAR, which comprises a preS1 domain of the hepatitis B virus envelope protein. Such a CRISPR/Cpf1 system includes the use of Cpf1, which is a single RNA guided endonuclease of a Class 2 CRISPR Cas9 system. As such, Cpf1, as a Class 2 effector, relies on single component effector proteins, such as Cas9 to mediate robust DNA interference with features that are distinct from Cas9. Cpf1 is a CRISPR-associated two-component RNA-programmable DNA nuclease. In some embodiments, the use of chemically modified CRISPR guide polynucleotides, such as RNAs, is contemplated for delivery by said cell or vesicle having a CAR, which comprises the preS1 domain of the hepatitis B virus envelope protein. Chemically-modified guide RNAs have been used in CRISPR-Cas genome editing in human primary cells (Mendel; A. et al., Nat Biotechnol. 2015 September; 33(9):985-9). Chemical modifications of guide RNAs can include modifications that confer nuclease resistance. Nucleases can be endonucleases, or exonucleases, or both. Some chemical modification, without limitations; include 2′-fluoro, 2′O-methyl; phosphorothioate dithiol 3′-3′ end linkage, 2-amino-dA, 5-methyl-dC; C-5 propynyl-C, or C-5 propynyl-U, morpholino. These examples are not meant to be limiting and other chemical modifications and variants and modifications of these exemplary embodiments are also contemplated.

“Chimeric receptor” as used herein refers to a synthetically designed receptor comprising a ligand binding domain of an antibody or other protein sequence that binds to a molecule associated with the disease or disorder and is linked via a spacer domain to one or more intracellular signaling domains of a cell such as a T cell or other receptors, such as a costimulatory domain. Chimeric receptor can also be referred to as artificial T cell receptors, chimeric T cell receptors, chimeric immunoreceptors, and chimeric antigen receptors (CARs). These receptors can be used e.g., to graft the specificity of the preS1 domain of the hepatitis B virus envelope protein onto a cell or vesicle, such as a T-cell, wherein transfer of their coding sequences is facilitated by viral vectors, such as a retroviral vector or a lentiviral vector, CARs are genetically engineered cell receptors, such as T-cell receptors designed to redirect cells, such as I-cells, to target cells that express specific cell-surface antigens, such as liver cells expressing NTCP. Cells such as T-cells can be removed from a subject and modified so that they can express receptors that can be specific for an antigen by a process called adoptive cell transfer. The cells, such as T-cells are reintroduced into the patient where they can then recognize and target an antigen. These CARs are engineered receptors that can graft a selected specificity onto an immune receptor cell. Due to the surprising effects of modifying the different components or domains of the CAR described herein, such as the epitope binding region (for example, preS1 domain or a portion thereof), spacer, transmembrane domain, and/or signaling domain), the components of the CAR are frequently distinguished throughout this disclosure in terms of independent elements.

T-cells” or “T lymphocytes” as used herein, can be from any mammalian species, preferably primate, including monkeys, dogs, or humans. In some alternatives the T-cells are allogeneic (from the same species but different donor) as the recipient subject; in some alternatives the T-cells are autologous (the donor and the recipient are the same); in some alternatives the T-cells are syngeneic (the donor and the recipients are different but are identical twins).

“Subject” or “patient,” as described herein, refers to any organism upon which the alternatives described herein may be used or administered, e.g., for experimental, diagnostic, prophylactic, and/or therapeutic purposes. Subjects or patients include, for example, animals. In some alternatives; the subject is mice, rats, rabbits, non-human primates; or humans. In some alternatives, the subject is a cow, sheep, pig, horse, dog, cat, primate or a human.

CARS Directed to NTCP Expressing Cells

Some embodiments provided herein relate to nucleic acids and polypeptides encoding a CAR and a cell or vesicle comprising said nucleic acids and polypeptides encoding said CAR, such as a T cell, and methods of using said cells or vesicle, as described herein, wherein said CAR comprises a preS1 domain of the hepatitis B virus envelope protein and, wherein said preS1 domain of said CAR comprises an amino acid sequence identity that is at least 90%, 91%, 92%, 93%, 94%, 95%, 96%, 97%, 98%, 99%, or 100% identical to:

(SEQ ID NO: 3)
GTNLSVTNPLGFFPDHQLDPAFGAN
SNNPDWDFNPNKDHWPDANKVG

Optionally, some embodiments provided herein relate to nucleic acids and polypeptides encoding a CAR and a cell or vesicle comprising said nucleic acids and polypeptides encoding said CAR, such as a T cell, and methods of using said cells or vesicles, as described herein, wherein said CAR comprises a preS1 domain of the hepatitis B virus envelope protein and, wherein said preS1 domain of said CAR comprises an amino acid sequence identity that is at least 90%, 91%, 92%, 93%, 94%, 95%, 96%, 97%, 98%, 99%; or 100% identical to SEQ ID NO. 3 and further comprises a signal peptide that is at least 90%, 91%, %, 93%, 94%, 95%, 96%, 97%; 98%, 99%, or 100% identical to:

(SEQ. ID. NO. 13)
MALPVTALLLPLALLLHAARP.

Some embodiments provided herein relate to nucleic acids and polypeptides encoding a CAR and a cell or vesicle comprising said nucleic acids and polypeptides encoding said CAR, such as a T cell, and methods of using said cells or vesicles, as described herein, wherein said CAR comprises a preS1 domain of the hepatitis B virus envelope protein, wherein said preS1 domain of said CAR comprises an amino acid sequence that has a sequence identity that is at least 90%, 91%, 92%, 93%, 94%, 95%, 96%, 97%, 98%, 99%, or 100% identical to (SEQ ID NO: 3), optionally further comprising the signal peptide of SEQ ID NO 13, and further comprising a peptide spacer domain, such as a CD8 hinge spacer that has a sequence identity that is at least 90%, 91%, 92%, 93%, 94%, 95%, 96%, 97%, 98%, 99%, or 100% identical to:

(SEQ ID NO: 4)
TTTPAPRPPTPAPTIASQPLSLRPE
ACRPAAGGAVHTRGLDFACD

In some alternatives, the peptide spacer is 45 amino acids or less but not less than 1 or 2 amino acids e.g., of SEQ ID NO 4. In some aspects, the polypeptide spacer chain may range in length, such as from a length of 3, 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239 or 240 amino acids or a length within a range defined by any two of the aforementioned lengths. A spacer can comprise any 45 amino acids or less but not 1 or 2 amino acids, for example, in any order to create a desirable length of polypeptide chain in a chimeric receptor, which includes the amino acids arginine, histidine, lysine, aspartic acid, glutamic acid, serine, threonine, asparagine, glutamine, cysteine, glycine, proline, alanine, valine, isoleucine, methionine, phenylalanine, tyrosine or tryptophan. In some alternatives, the spacer resides between the preS1 binding domain and the transmembrane region of the chimeric receptor. A spacer may also be customized, selected, configured for, or optimized for a desired length so as to improve binding of the preS1 domain or ligand binding domain to the target cell, which may increase cytotoxic efficacy. In some alternatives, the linker or spacer between the preS1 domain or ligand binding domain and the transmembrane domain can be 25 to 55 amino acids in length (e.g., at least, equal to 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids or a length within a range defined by any two of the aforementioned lengths). Example spacers include IgG4 hinge alone, IgG4 hinge linked to CH2 and CH3 domains, or IgG4 hinge linked to the CH3 domain.

Some embodiments provided herein relate to nucleic acids and polypeptides encoding a CAR and a cell or vesicle comprising said nucleic acids and polypeptides encoding said CAR, such as a T cell, and methods of using said cells or vesicles, as described herein, wherein said CAR comprises a preS1 domain of the hepatitis B virus envelope protein, wherein said preS1 domain of said CAR comprises an amino acid sequence that has a sequence identity that is at least 90%, 91%, 92%, 93%, 94%, 95%, 96%, 97%, 98%, 99%, or 100% identical to (SEQ ID NO: 3), optionally further comprising the signal peptide of SEQ TD NO 13, and further comprising a peptide spacer domain, such as a CD8 hinge spacer that has a sequence identity that is at least 90%, 91%, 92%, 93%, 94%, 95%, 96%, 97%, 98%, 99%, or 100% identical to SEQ ID NO: 4 and further comprising a transmembrane domain, such as a CD8 transmembrane domain e.g., transmembrane domain that has a sequence identity that is at least 90%, 91%, 92%, 93%, 94%, 95%, 96%, 97%, 98%, 99%, or 100% identical to:

(SEQ ID NO: 5)
IYIWAPLAGTCGVLLLSLVITLYC.

In some embodiments, a signaling domain of a CAR, such as primary signaling domain or costimulatory domain, includes an intracellular or cytoplasmic domain of a protein or a receptor protein that interacts with components within the interior of the cells and is capable of relaying or participating in relaying a signal. Such interactions in some aspects can occur through the intracellular domain communicating via specific protein-protein or protein-ligand interactions with an effector molecule or an effector protein, which in turn can send the signal along a signal chain to its destination. In some embodiments, the signaling domain includes a co-stimulatory domain. In some aspects, the costimulatory domain includes a signaling moiety that provides to cells such as T cells a signal, which in addition to the primary signal provided by for instance the CD3 zeta chain of the TCR/CD3 complex, enhances a response such as a T-cell effector response, such as, for example, an immune response, activation, proliferation, differentiation, cytokine secretion, cytolytic activity, perforin or granzyme activity and the like. In some embodiments, the intracellular signaling domain and/or the co-stimulatory domain can include all or a portion of, but is not limited to, CD27, CD28, 4-1BB, OX40, CD30, CD40, ICOS, lymphocyte function-associated antigen-1 (LFA-1), CD2, CD7, LIGHT, NKG2C, or B7-H3, or a ligand that specifically binds with CD83.

Accordingly, some embodiments provided herein relate to nucleic acids and polypeptides encoding a CAR and a cell or vesicle comprising said nucleic acids and polypeptides encoding said CAR, such as a T cell, and methods of using said cells or vessicles, as described herein, wherein said CAR comprises a preS1 domain of the hepatitis B virus envelope protein, wherein said preS1 domain of said CAR comprises an amino acid sequence that has a sequence identity that is at least 90%, 91%, 92%, 93%, 94%, 95%, 96%, 97%, 98%, 99%, or 100% identical to (SEQ ID NO: 3), optionally further comprising the signal peptide of SEQ ID NO 13, and further comprising a peptide spacer domain, such as a CD8 hinge spacer that has a sequence identity that is at least 90%, 91%, 92%, 93%, 94%, 95%, 96%, 97%, 98%, 99%, or 100% identical to SEQ TD NO: 4 and further comprising a transmembrane domain, such as a CD8 transmembrane domain e.g., transmembrane domain that has a sequence identity that is at least 90%, 91%, 92%, 93%, 94%, 95%, 96%, 97%, 98%, 99%, or 100% identical to SEQ ID NO: 5 and further comprising at least one costimulatory domain that is at least 90%, 91%, 92%, 93%, 94%, 95%, 96%, 97%, 98%, 99%, or 100% identical to:

a CD28 costimulatory domain:

(SEQ ID NO. 6)
RSKRSRLLHSDYMNMTPRRPGPTRKHYQPYAPPRDFAAYRS;

a 4-1BB costimulatory domain

(SEQ ID NO. 7)
KRGRKKLLYIFKQPFMRPVQTTQEEDGCSCRFPEEEEGGCEL;

or

a CD3 zeta costimulatory domain

(SEQ ID NO. 8)
RVKFSRSADAPAYKQGQNQLYNELNLGRREEYDVLDKRRGRD
PEMGGKPRRKNPQEGLYNELQKDKMAEAYSEIGMKGERRRGK
GHDGLYQGLSTATKDTYDALHMQALPPR.

Preferable embodiments provided herein relate to nucleic acids and polypeptides encoding a CAR and a cell or vesicle comprising said nucleic acids and polypeptides encoding said CAR, such as a T cell, and methods of using said cells or vesicles, as described herein, wherein said CAR comprises a preS1 domain of the hepatitis B virus envelope protein, wherein said preS1 domain of said CAR comprises an amino acid sequence that has a sequence identity that is at least 90%, 91%, 92%, 93%, 94%, 95%, 96%, 97%, 98%, 99%, or 100% identical to (SEQ ID NO: 3), optionally further comprising the signal peptide of SEQ ID NO 13, and further comprising a peptide spacer domain, such as a CD8 hinge spacer that has a sequence identity that is at least 90%, 91%, 92%, 93%, 94%, 95%, 96%, 97%, 98%, 99%, or 100% identical to SEQ ID NO: 4 and further comprising a transmembrane domain, such as a CD8 transmembrane domain e.g., transmembrane domain that has a sequence identity that is at least 90%, 91%, 92%, 93%, 94%, 95%, 96%, 97%, 98%, 99%, or 100% identical to SEQ ID NO: 5 and further comprising CD28, 4-1BB, and CD3 zeta costimulatory domains that are at least 90%, 91%, 92%, 93%, 94%, 95%, 96%, 97%, 98%, 99%, or 100% identical to SEQ ID Nos: 6-8.

Additional embodiments provided herein relate to nucleic acids and polypeptides encoding a CAR and a cell or vesicle comprising said nucleic acids and polypeptides encoding said CAR, such as a T cell, and methods of using said cells or vesicles, as described herein, wherein said CAR comprises a preS1 domain of the hepatitis B virus envelope protein, wherein said preS1 domain of said CAR comprises an amino acid sequence that has a sequence identity that is at least 90%, 91%, 92%, 93%, 94?, 95%, 96%, 97%, 98%, 99%, or 100% identical to (SEQ ID NO: 3), optionally further comprising the signal peptide of SEQ ID NO 13, and further comprising a peptide spacer domain, such as a CD8 hinge spacer that has a sequence identity that is at least 90%, 91%, 92%, 93%, 94%, 95%, 96%, 97%, 98%, 99%, or 100% identical to SEQ ID NO: 4 and further comprising a transmembrane domain, such as a CD8 transmembrane domain e.g., transmembrane domain that has a sequence identity that is at least 90%, 91%, 92%, 93%, 94%, 95%, 96%, 97%, 98%, 99%, or 100% identical to SEQ ID NO: 5 and further comprising at least one costimulatory domain that is at least 90%, 91%, 92%, 93%, 94%, 95%, 96%, 97%, 98%, 99%, or 100% identical to any one of SEQ ID Nos 6-8, preferably comprising CD28, 4-1BB, and CD3 zeta costimulatory domains that are at least 90%, 91%, 92%, 93%, 94%, 95%, 96%, 97%, 98%, 99%, or 100% identical to SEQ ID Nos: 6-8, and further comprising a cleavage sequence such as a T2A cleavage sequence that has at least 90%, 91%, 92%, 93%, 94%, 95%, 96%, 97%, 98%, 99%, or 100% sequence identity to:

(SEQ ID NO. 9)
EGRGSLLTCGDVEENPGP.

More embodiments provided herein relate to nucleic acids and polypeptides encoding a CAR and a cell or vesicle comprising said nucleic acids and polypeptides encoding said CAR, such as a T cell, and methods of using said cells or vesicles, as described herein, wherein said CAR comprises a preS1 domain of the hepatitis B virus envelope protein, wherein said preS1 domain of said CAR comprises an amino acid sequence that has a sequence identity that is at least 90%, 91%, 92%, 93%, 94%, 95%, 96%, 97%, 98%, 99%, or 100% identical to (SEQ ID NO: 3), optionally further comprising the signal peptide of SEQ ID NO 13, and further comprising a peptide spacer domain, such as a CD8 hinge spacer that has a sequence identity that is at least 90%, 91%, 92%, 93%, 94%, 95%, 96%, 97%, 98%, 99%, or 100% identical to SEQ ID NO: 4 and further comprising a transmembrane domain, such as a CD8 transmembrane domain e.g., transmembrane domain that has a sequence identity that is at least 90%, 91%, 92%, 93%, 94%, 95?, 96%, 97%, 98%, 99%, or 100% identical to SEQ ID NO: 5 and further comprising at least one costimulatory domain that is at least 90%, 91%, 92%, 93%, 94%, 95%, 96%, 97%, 98%, 99%, or 100% identical to any one of SEQ ID Nos 6-8, preferably comprising CD28, 4-1BB, and CD3 zeta costimulatory domains that are at least 90%, 91%, 92%, 93%, 94%, 95%, 96%, 97%, 98%, 99%, or 100% identical to SEQ ID Nos: 6-8, and further comprising a cleavage sequence such as a T2A cleavage sequence that has at least 90%, 91%, 92%, 93%, 94%, 95%, 96%, 97%, 98%, 99%, or 100% sequence identity to SEQ ID NO. 9 and further comprising a marker sequence such as an EGFP marker sequence that is at least 90%, 91%, 92%, 93%, 94%, 95%, 96%, 97%, 98%, 99%, or 100% identical to:

(SEQ ID NO 10)
MVSKGEELFTGVVPILVELDGDVNGH
KFSVSGEGEGDATYGKLTLKFICTTG
KLPVPWPTLVTTLTYGVQCFSRYPDH
MKQHDFFKSAMPEGYVQERIIFEKDD
GNYKTRAEVKFEGDTLVNRIELKGID
FKEDGNTLGHKLEYNYNSHNVYIMAD
KQKNGIKVWKIRHNIEDGSVQLADHY
QQNTPIGDGPVLLPDNHYLSTQSALS
KDPNEKRDHMVLLEFVTAAGITLGMD
ELYK.

Preferred nucleic acids and polypeptides encoding a CAR and cells or vesicles comprising said nucleic acids and polypeptides encoding said CAR, such as a T cell, and methods of using said cells or vesicles, as described herein, include an amino acid sequence having at least 90%, 91%, 92%, 93%, 94%, 95%, 96%, 97%, 98%, 99%, or 100% sequence identity to all of the elements set forth in SEQ ID NOs: 3-10 assembled in tandem (e.g., see FIG. 1), optionally further comprising the signal peptide of SEQ ID NO 13. Preferred nucleic acids and polypeptides encoding a CAR and cells or vesicles comprising said nucleic acids and polypeptides encoding said CAR, such as a T cell, and methods of using said cells, as described herein, e.g., have at least 90%, 91%, 92%, 93%, 94%, 95%, 96%, 97%, 98%, 99%, or 100% sequence identity to the construct set forth herein as signal peptide-PreS1-CD8 hinge-CD8 Transmembrane-CD28-4-1BB-CD3zeta-T2A-EGFP provided by the sequence:

(SEQ ID NO 11)
MALPVTALLLPLALLLHAARPGTNLSV
PNPLGFFPDHQLDPAFGANSNNPDWDF
NPNKDHWPDANKVGTTTPAPRPPTPAP
TIASQPLSLRPEACRPAAGGAVHTRGL
DFACDIYIWAPLAGTCGVLLLSLVITL
YCRSKRSRLLHSDYMNMTPRRPGPTRK
HYQPYAPPRDFAAYRSKRGRKKLLYIF
KQPFMRPVQTTQEEDGCSCRFPEEEEG
GCELRVKFSRSADAPAYKQGQNQLYNE
LNLGRREEYDVLDKRRGRDPEMGGKPR
RKNPQEGLYNELQKDKMAEAYSEIGMK
GERRRGKGHDGLYQGLSTATKDTYDAL
HMQALPPREGRGSLLTCGDVEENPGPM
VSKGEELFTGVVPILVELDGDVNGHKF
SVSGEGEGDATYGKLTLKFICTTGKLP
VPWPTLVTTLTYGVQCFSRYPDHMKQH
DFFKSAMPEGYVQERTIFFKDDGNYKT
RAEVKFEGDTLVNRIELKGIDFKEDGN
ILGHKLEYNYNSHNVYIMADKQKNGIK
VNFKIRHNIEDGSVQLADHYQQNTPIG
DGPVLLPDNHYLSTQSALSKDPNEKRD
HMVLLEFVTAAGITLQMDELYK.

Embodiments of the present disclosure also concern nucleic acids encoding the CARs described herein and cells or vesicles comprising said nucleic acids. Preferred embodiments comprise a nucleic acid encoding SEQ ID NO 11 and a cell or vesicle, such as a T cell, comprising a nucleic acid encoding SEQ ID NO 11 or a nucleic acid having at least 90%, 91%, 92%, 93%, 94%, 95%, 96%, 97%, 98%, 99%, or 100% sequence identity to a nucleic acid encoding SEQ ID NO 11 or a cell comprising a nucleic acid having at least 90%, 91%, 92%, 93%, 94%, 95%, 96%, 97%, 98%, 99%, or 100% sequence identity to a nucleic acid encoding SEQ ID NO 11. Preferred embodiments comprise, for instance, a nucleic acid having at least 90%, 91%, 92%, 93%, 94%, 95%, 96%, 97%, 98%, 99%, or 100% sequence identity to a nucleic acid encoding the construct set forth herein as signal peptide-PreS1-CD8 hinge-CD8 Transmembrane-CD28-4-1BB-CD3zeta-T2A-EGFP such as that provided by SEQ ID NO 1.2:

(SEQ ID NO. 12)
ATGGCCTTACCAGTGACCGCCTTGCTCC
TGCCGCTGGCCTTGCTGCTCCACGGCG
CCAGGCCGGGCACAAATCTGAGCGTGC
CAAATCCTCTGGGATTCTTCCCAGACC
ACCAGCTGGATCCTGCCTTCGGCGCCA
ACAGCAACAACCCCGACTGGGACTTCA
ACCCCAACAAGGACCATTGGCCTGACG
CCAACAAAGTGGGCACCACGACGCCAG
CGCCGCGACCACCAACACCGGCGCCCA
CCATCGCGTCGCAGCCCCTGTCCCTGC
GCCCAGAGGCGTGCCGGCCAGCGGCGG
GGGGCGCAGTGCACACGAGGGGGCTGG
ACTTCGCCTGTGATATCTACATTTGGG
CCCCTCTGGCTGGTACTTGCGGGGTCC
TGCTGCTTTCACTCGTGATCACTCTTT
ACTGTAGGAGCAAGCGGAGCAGACTGC
TGCACAGCGACTACATGAACATGACCC
CCCGGAGGCCTGGCCCCACCCGGAAGC
ACTACCAGCCCTACGCCCCTCCCAGGG
ATTTCGCCGCCTACCGGAGCAAGCGCG
GTCGGAAGAAGCTGCTGTACATCTTTA
AGCAACCCTTCATGAGGCCTGTGCAGA
CTACTCAAGAGGAGGACGGCTGTTCAT
GCCGGTTCCCAGAGGAGGAGGAAGGCG
GCTGCGAACTGAGAGTGAAGTTCAGCA
GGAGCGCAGACGCCCCCGCGTACAAGC
AGGGCCAGAACCAGCTCTATAACGAGC
TCAATCTAGGACGAAGAGAGGAGTACG
ATGTTTTGGACAAGAGACGTGGCCGGG
ACCCTGAGATGGGGGGAAAGCCGAGAA
GGAAGAACCCTCAGGAAGGCCTGTACA
ATGAACTGCAGAAAGATAAGATGGCGG
AGGCCTACAGTGAGATTGGGATGAAAG
GCGAGCGCCGGAGGGGCAAGGGGCACG
ATGGCCTTTACCAGGGTCTCAGTACAG
CCACCAAGGACACCTACGACGCCCTTC
ACATGCAGGCCCTGCCCCCTCGCGAGG
GCAGGGGAAGTCTTCTAACATGCGGGG
ACGTGGAGGAAAATCCCGGCCCCATGG
TGAGCAAGGGCGAGGAGCTGTTCACCG
GGGTGGTGCCCATCCTGGTCGAGCTGG
ACGGCGACGTAAACGGCCACAAGTTCA
GCGTGTCCGGCGAGGGCGAGGGCGATG
CCACCTACGGCAAGCTGACCCTGAAGT
TCATCTGCACCACCGGCAAGCTGCCCG
TGCCCTGGCCCACCCTCGTGACCACCC
TGACCTACGGCGTGCAGTGCTTCAGCC
GCTACCCCGACCACATGAAGCAGCACG
ACTTCTTCAAGTCCGCCATGCCCGAAG
GCTACGTCCAGGAGCGCACCATCTTCT
TCAAGGACGACGGCAACTACAAGACCC
GCGCCGAGGTGAAGTTCGAGGGCGACA
CCCTGGTGAACCGCATCGAGCTGAAGG
GCATCGACTTCAAGGAGGACGGCAACA
TCCTGGGGCACAAGCTGGAGTACAACT
ACAACAGCCACAACGTCTATATCATGG
CCGACAAGCAGAAGAACGGCATCAAGG
TGAACTTCAAGATCCGCCACAACATCG
AGGACGGCAGCGTGCAGCTCGCCGACC
ACTACCAGCAGAACACCCCCATCGGCG
ACGGCCCCGTGCTGCTGCCCGACAACC
ACTACCTGAGCACCCAGTCCGCCCTGA
GCAAAGACCCCAACGAGAAGCGCGATC
ACATGGTCCTGCTGGAGTTCGTGACCG
CCGCCGGGATCACTCTCGGCATGGACG
AGCTGTACAAGTAA.

### Methods of Therapy

Some embodiments provided herein include compositions, such as a cell or vesicle, preferably cells, comprising one or more CARs, which comprise the preS1 domain of the hepatitis B virus envelope protein, and nucleic acids and polypeptides encoding said CARs. Additional embodiments include methods of using said cells, preferably T cells, comprising said CARs to interact or to bind to NTCP expressing cells, such as liver cells, and methods of modulating the interaction or binding of said cells comprising said one or more CARs, preferably cells, to said NTCP expressing cells by administering or providing an antibody or binding fragment thereof, such as an scFv or Fab fragment, or peptide, which binds to or interacts with said preS1 domain of said. CARs. In some embodiments, cells, preferably T cells, comprising one or more CARs, which comprise the preS1 domain of the hepatitis B virus envelope protein, are administered to a subject having NTCP expressing cells, preferably a human and, optionally, subsequent to said administration, an antibody or binding fragment thereof, such as an scFv or Fab fragment, or peptide, which binds to or interacts with said preS1 domain of said CARs is administered to said subject. In some embodiments, said cells or vesicles comprising a CAR, which comprises the preS1 domain, further comprises a nucleic acid, protein, or drug, which is desired for deliver to said NTCPO expressing cells, such as liver cells, and said nucleic acids can encode, for example a molecule, which is desired for expression or a CRISPR/CAS system.

In some such embodiments, administering the antibody or binding fragment thereof, such as an scFv or Fab fragment, or peptide, which binds to or interacts with said preS1 domain of said CARs, to the subject, preferably a human, modulates the activity of the cells or vesicles comprising the CARs e.g., T cells. For instance, the antibody or binding fragment thereof, such as an scFv or Fab fragment, or peptide, which binds to or interacts with said preS1 domain of said CARs, can bind to the cells or vesicles comprising the CARs e.g., T cells without invoking substantial signaling in the cells. In some embodiments, the presence of the antibody or binding fragment thereof, such as an scFv or Fab fragment, or peptide, which binds to or interacts with said preS1 domain of said CARs, reduces an effector function of the cells comprising the CARs e.g., cells, as compared to effector functions in the absence of the antibody or binding fragment thereof, such as an scFv or Fab fragment, or peptide, which binds to or interacts with said preS1 domain of said CARs.

In some embodiments, administration of the antibody or binding fragment thereof, such as an scFv or Fab fragment, or peptide, which binds to or interacts with said preS1 domain of said CARs, reduces cytokine production from the cells comprising said CARs, e.g., T cells, as compared to the cytokine production from cells comprising said CARs, e.g., T cells, in the absence of the antibody or binding fragment thereof, such as an scFv or Fab fragment, or peptide, which binds to or interacts with said preS1 domain of said. CARs. Accordingly, in some embodiments, the presence of the antibody or binding fragment thereof, such as an scFv or Fab fragment, or peptide, which binds to or interacts with said preS1 domain of said CARs, in said subject, preferably a human, reduces undesirable side effects of the cells comprising the CARs e.g., T cells, such as cytokine release syndrome in said subject, as compared to side effects in the absence of the antibody or binding fragment thereof, such as an scFv or Fab fragment, or peptide, which binds to or interacts with said preS1 domain of said CARs in said subject.

In some embodiments, the antibody or binding fragment thereof, such as an scFv or Fab fragment, used to modulate said activities of the cells or vesicles comprising the CARS e.g., T cells, in said subject, preferably a human, are e.g., monoclonal or polyclonal antibodies or binding fragments thereof, such as scFv or Fab fragments, preferably humanized, wherein said monoclonal or polyclonal antibodies or binding fragments thereof bind to epitopes present on the preS1 domain of the hepatitis B virus envelope protein. Preferred epitopes, which are bound by said monoclonal or polyclonal antibodies or binding fragments thereof, such as scFv or Fab fragments, preferably humanized, include residues 19-33 of the pre-S1 domain. A suitable monoclonal antibody specific to this epitope (mAb 2D3) is described in Yan et al., “Sodium taurocholate co-transporting polypeptide is a functional receptor for human hepatitis B and D virus”, eLife. 2012; 1: e00049, published online 2012 Nov. 13, hereby expressly incorporated by reference in its entirety. Additional monoclonal antibodies, which bind to the preS1 domain and from which scFv and Fab fragments can be generated or are commercially available e.g., under catalog number ABIN934315 from Antibodies Online. A variety of anti-preS1 antibodies and binding fragments thereof, which are suitable in the methods described herein are also available through Creative Biolabs e.g., Recombinant Humanized Anti-HBV preS1 Antibody (HzKR127), Recombinant Anti-HBV preS1 Antibody (MOB-437), Recombinant Humanized Anti-HBV preS1 Antibody scFv Fragment (HzKR127)(PSBC-054), Recombinant Anti-HBV preS1 Antibody scFv Fragment (MOB-437-S(P)), Recombinant Anti-HBV preS1 Antibody scFv Fragment (MOB-799-S(P)), Recombinant Human Anti-HBV preS1 Antibody Fab Fragment (2H5-A14) (PFBC-406), Recombinant Human Anti-HBV preS1 Antibody Fab Fragment (MHH-799-RE)), Recombinant Human Anti-HBV preS1 Antibody scFv Fragment (MHH-799-S(P)), or Recombinant Anti-HBV preS1 Antibody Fab Fragment (MOB-437-F(E)).

In some embodiments, the peptide, which binds to or interacts with said preS1 domain of said CARs, thereby modulating said activities of the cells comprising the CARs, e.g., T cells, in said subject, preferably a human, are peptides encompassing a domain of NTCP, which interacts with or binds to the preS1 domain of the hepatitis B virus envelope protein. In some such embodiments, the peptide is or comprises the full-length NTCP protein the sequence of which e.g., is provided by NCBI as Q14973.1 as:

```
(SEQ ID NO. 1)
    1 meahnasapf nftlppnfgk rptdlalsvi
        lvfmlffiml slgctmefsk ikahlwkpkg
  61 laialvaqyg impltafvlg kvfrlkniea
        lailvcgcsp ggnlsnvfsl amkgdmnlsi
121 vmttcstfca lgmmplllyi ysrgiydgdl
        kdkvpykgiv islvlvlipc tigivlkskr
181 pqymryvikg gmiiillcsv avtvlsainv
        gksimfamtp lliatsslmp figfllgyvl
241 salfclngrc rrtvsmetgc qnvqlcstil
        nvafppevig plfffpllym ifqlgeglll
301 iaifwcyekf ktpkdktkmi ytaatteeti
        pgalgngtyk gedcspcta.
```

In additional embodiments, full-length NTCP protein can be modified to confer stability e.g., by pegylation or conjugation to albumin or a mutant, variant or derivative thereof. In more embodiments, the peptide, which binds to or interacts with said preS1 domain of said CARs, thereby modulating said activities of the cells comprising the CARs, e.g., T cells, in said subject, preferably a human, are peptides comprising residues 157-165 of a human NTCP protein sequence, which can be in some alternatives comprise the amino acid sequence: KGIVISLVL (SEQ ID NO. 2) and, which can also be modified e.g., by pegylation or conjugation to an albumin or derivative thereof.

In some embodiments, the cells comprising the preS1-containing CARs set forth herein are white blood cells, NK cells, or precursor T cells. In some embodiments, the cells comprising the preS1-containing CARs set forth herein are hematopoietic stem cells. In some embodiments, the cells comprising the preS1-containing CARs set forth herein are CD8+ T cytotoxic lymphocyte cells selected from the group consisting of naïve CD8+ T cells, central memory CD8+ T cells, effector memory CD8+ T cells and bulk CD8+ T cells. In some embodiments, the cells comprising the preS1-containing CARs set forth herein are CD4+ T helper lymphocyte cells selected from the group consisting of naïve CD4+ T cells, central memory CD4+ T cells, effector memory CD4+ T cells, and bulk CD4+ T cells.

More embodiments concern methods of treating, inhibiting or ameliorating a liver disease or a condition associated therewith such as a liver cancer e.g., hepatocellular carcinoma, or an infection of liver cells induced by a pathogen, such as a viral pathogen in a subject, preferably a human. In some embodiments, the liver disease or condition associated therewith is chronic in said subject, preferably a human. Some approaches are practiced by administering a plurality of cells or vesicles, preferably T cells, which have been engineered to have CARs, which comprise a preS1 domain of a hepatitis B virus envelope protein to a subject, preferably a human, which has a liver disease or a condition associated therewith such as a liver cancer e.g., hepatocellular carcinoma, or an infection of liver cells induced by a pathogen, such as a viral pathogen and, optionally, subsequent to said administration, an antibody or binding fragment thereof, such as an scFv or Fab fragment, or peptide, which binds to or interacts with said preS1 domain of said CARs is administered to said subject. In some embodiments, the cells or vesicles further comprise a nucleic acid or protein or drug, which is desired to be delivered to the NTCP-expressing cells. In some embodiments, the cells or vesicles comprise CRISPR/CAS system, which can be delivered to the NTCP-expressing cells. In some of these therapeutic embodiments, the antibody or binding fragment thereof, such as an scFv or Fab fragment, used to modulate said activities of the cells comprising the CARs e.g., T cells, in said subject, preferably a human, are e.g., monoclonal or polyclonal antibodies or binding fragments thereof, such as scFv or Fab fragments, preferably humanized, wherein said monoclonal or polyclonal antibodies or binding fragments thereof bind to epitopes present on the preS1 domain of the hepatitis B virus envelope protein. Exemplary antibodies and binding fragments thereof useful in these therapeutic methods include mAb 2D3, anti-preS1 antibody available under catalog number ABIN934315, Recombinant Humanized Anti-HBV preS1 Antibody (HzKRI27), Recombinant Anti-HBV preS1 Antibody (MOB-437), Recombinant Humanized Anti-HBV preS1 Antibody scFv Fragment (HzKR127)(PSBC-054), Recombinant Anti-HBV preS1 Antibody say Fragment (MOB-437-S(P)), Recombinant Anti-HBV preS1 Antibody scFv Fragment (MOB-799-S(P)), Recombinant Human Anti-HBV preS1 Antibody Fab Fragment (2H5-A14) (PFBC-406), Recombinant Human Anti-HBV preS1 Antibody Fab Fragment (MHH-799-F(E)), Recombinant Human Anti-HBV preS1 Antibody scFv Fragment (MHH-799-S(P)), or Recombinant Anti-HBV preS1 Antibody Fab Fragment (MOB-437-F(E)). In some of these therapeutic embodiments, the peptide, which binds to or interacts with said preS1 domain of said CARs, thereby modulating said activities of the cells comprising the CARs, e.g., T cells, in said subject, preferably a human, are peptides encompassing a domain of NTCP, which interacts with or binds to the preS1 domain of the hepatitis B virus envelope protein. Exemplary peptides useful in these therapeutic methods include the full-length NTCP protein, e.g., SEQ II) NO 1, which can be modified e.g., by pegylation or conjugation to an albumin or a variant or mutant or derivative thereof, or a preS1 domain binding fragment of NTCP, such as peptides comprising residues 157-165 of a human NTCP protein sequence, which can be in some alternatives comprise the amino acid sequence of SEQ ID NO. 2, which can also be modified e.g., by pegylation or conjugation to an albumin or variant, or mutant or derivative thereof.

### Example

The CAR-PreS1 construct shown in FIG. 1 was used to transfect commercially available PBMC. The T cells were expanded by culturing for 6 days in AIMV medium+2% Human AB serum+50 ng/ml OKT3+600 IU/ml IL2. By the sixth day, the IL2 concentration had increased to 1000 IU/ml. On day 7, five million T cells were electroporated with either mRNA encoding the CAR-preS1 construct shown in FIG. 1 or a “Mock”) construct of 5 ug pVAX.10 mRNA. 18 h post-electroporation the CAR receptor expression was assessed by measuring GFP+. To access the functionality of the cells comprising the preS1 domain CARs, on the day of electroporation, 100,000 target cells were seeded in a flat-bottomed 96 well plate and 24 hours post-electroporation, 100,000 live effector cells were added according to the following layout:

| | Negative Controls | Samples | |
|---|---|---|---|
| Mock T cells | HepG2 | Hep62-NTCP | Huh7-NTCP |
| pVAX.10 T cells | HepG2 | Hep62-NTCP | Huh7-NTCP |

The cells were cultured for 6 hours in the presence of antibody against CD107a, GolgiPlug and GolgiStop (1:1000) and at the end of the co-culture, the cells were stained for IFN-γ and TNF alpha.

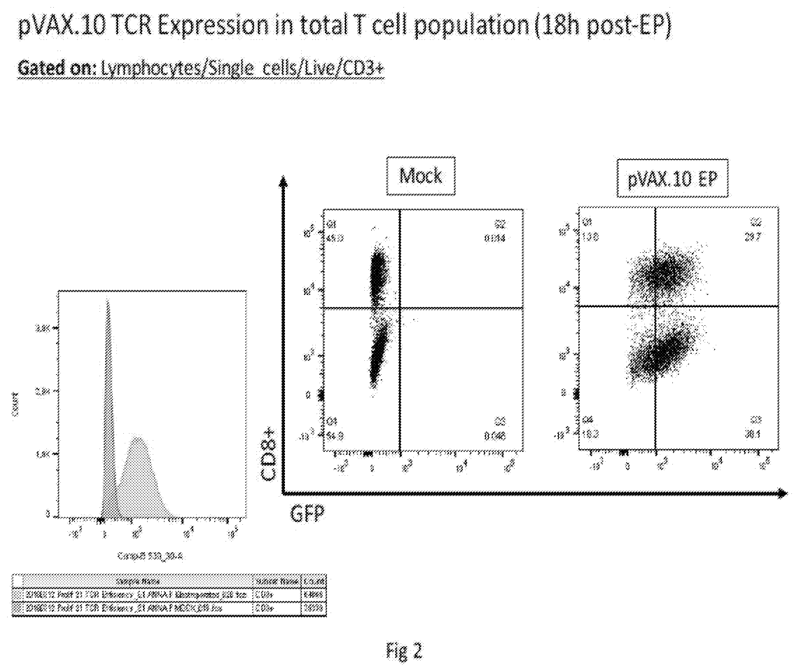

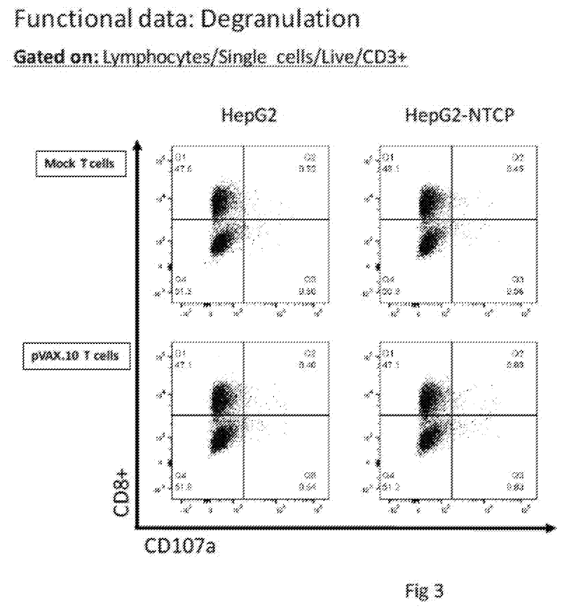

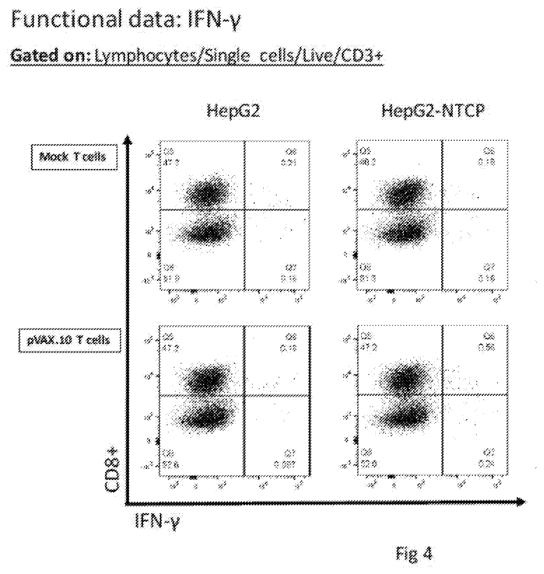

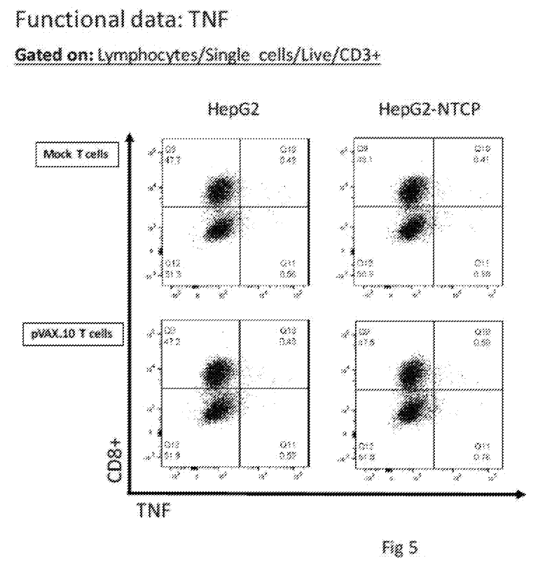

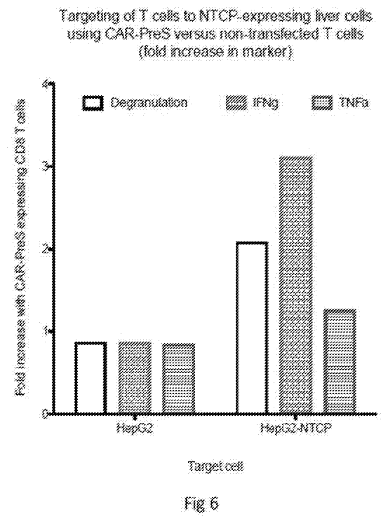

Transfection with CAR-PreS1 domain mRNA induced GFP expression in 30% of CD3+ T cells (FIG. 2). The T cells were then co-cultured with HepG2 cells HepG2 cells expressing or lacking NTCP receptor to which PreS1 binds, and the T cells were analyzed for expression of CD107 (FIG. 3), IFN gamma (FIG. 4) and TNF alpha (FIG. 5). This showed that the presence of NTCP on the hepatocytes induced an 2-fold increase in degranulation and a 3-fold increase in IFN gamma expression (FIG. 6). This evidence demonstrates that T cells transfected with a CAR-PreS1 molecule specifically recognize hepatocytes expressing NTCP. Thus, the CAR-PreS molecule can efficiently target T cells to NTCP-expressing hepatocytes.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

```
SEQUENCES
Amino acid sequence encoding NTCP
protein (NCBI as 014973.1)
(SEQ ID NO. 1)
    1 meahnasapf nftlppnfgk rptdlalsvi
        lvfmlffiml slgctmefsk ikahlwkpkg
  61 laialvaqyg impltafvlg kvfrlkniea
        lailvcgcsp ggnlsnvfsl amkgdmnlsi
121 vmttcstfca lgmmplllyi ysrgiydgdl
        kdkvpykgiv islvlvlipc tigivlkskr
181 pqymryvikg gmiiillcsv avtvlsainv
        gksimfamtp lliatsslmp figfllgyvl
241 salfclngrc rrtvsmetgc qnvqlcstil
        nvafppevig plfffpllym ifqlgeglll
301 iaifwcyekf ktpkdktkmi ytaatteeti
        pgalgngtyk gedcspcta
Amino acid sequence encoding residues
157-163 of a human NTCP protein
sequence
(SEQ ID NO. 2)
KGIVISLVL
Amino acid sequence encoding Pre
S1 domain sequence
(SEQ ID NO. 3)
GTNLSVPNPLGFFPDHQLDPAFGANSNN
PDWDFNPNKDHWPDANKVG
Amino acid sequence encoding CD8
hinge spacer
(SEQ ID NO. 4)
TTTPAPRPPTPAPTTASQpLSLRPEACRP
AAGGAVHIRGLDFACD
Amino acid sequence encoding CD8
Transmembrane domain
(SEQ ID NO. 5)
IYIWAPLAGTCGVLLLSLVITLYC
Amino acid sequence encoding CD28
costimulatory domain
(SEQ ID NO. 6)
RSKRSRLLHSDYMNMTPRRPGPTRKHYQPYAPPRD
FAAYRS
Amino acid sequence encoding 4-1BB
costimulatory domain
(SEQ ID NO. 7)
KRGRKKLLYIFKQPFMRPVQTTQEEDGCSCRFPEEE
EGGCEL
Amino acid sequence encoding CD3 zeta
costimulatory domain
(SEQ ID NO. 8)
RVKFSRSADAPAYKQGQNQLYNELNLGRREEYDVLD
KRRGRDPEMGGKPRRKNPQEGLYNELQKDKAIAEAY
SEIGMKGERRRGKGIIDGLYQGLSTATKDTYDALHM
QALPPR
Amino acid sequence encoding T2A
cleavage domain
(SEQ ID NO. 9)
EGRGSLLTCGDVEENPGP
Amino acid sequence encoding EGFP
marker domain
(SEQ ID NO. 10)
MVSKGEELFTGVVPILVELDGDVNGIIKESVSGEG
EGDATYGKLTLKFICTTGKLPVPWPTLVTTLTYGV
QCFSRYPDHMKQHDFFKSAMPEGYYQERTIFFKDD
GNYKTRAEVKFEGDTLVNRIELKGIDFKEDGNILG
HKLEYNYNSHNVYIMADKQKNGIKVNFKIRHNIED
GSVQLADHYQQNTPIGDGFVLLPDNHYLSTQSALS
KDPNEKRDHMVLLEFVTAAGITLGMDELYK
Amino acid sequence encoding signal
peptide-Pre S1-CD8 hinge-CD8
Transmembrane-CD28-4-1BB-
CD3zeta-T2A-EGFP
(SEQ ID NO. 11)
MALPVTALLLPLALLLHAARPGTNLSVPNPLGFFP
DFTQLDPAFGANSNNPDWDFNPNKDHWPDANKVGT
TTPAPRPPTPAPTIASQPLSLRPEACRPAAGGAVH
TRGLDFACDIYIWAPLAGTCGVLLLSLMTLYCRSK
RSRLLHSDYMNMIPRRPGPTRKHYQPYAPPRDFAAY
RSKRGRKKLLYIFKQPFMRPVQTTQEEDGCSCRFP
EEEEGGCELRVKFSRSADAPAYKQGQNQLYNELNL
GRREEYDVLDKRRGRDPEMGGKPRRKNPQEGLYNE
LQKDKMAEAYSEIGMKGERRRGKGHDGLYQGLSTA
TKDTYDALHMQALPPREGRGSLLTCGDVEENPGPM
VSKGEELFTGVVPILVELDGDVNGHKFSVSGEGEG
DATYGKLTLKFICTTGKLPVPWPTLVTTLTYGVQC
FSRYPDHMKQHDFFKSAMPEGYVQERTIFFKDDGN
YKTRAEVKFEGDTLVNRIELKGIDFKEDGNILGHK
LEYNYNSHNVYIMADKQKNGIKVNFKIRHNIEDGS
VQLADFIYQQNTPIGDGPVLLPDNHYLSTQSALSK
DPNEKRDHMVLLEFVTAAGITLGMDELYK
Nucleic acid sequence encoding EF-1
Alpha promoter-signal peptide-Pre S1-
CD8 hinge-CD8 Transmembrane-CD28-4-
1BB-CD3zeta-T2A-EGFP
(SEQ ID NO. 12)
ATGGCCTTACCAGTGACCGCCTTGCTCCTGCCGCTG
GCCTTGCTGCTCCACGCCGCCAGGCCGGGCACAAA
TCTGAGCGTGCCAAATCCTCTGGGATTCTTCCCAG
ACCACCAGCTGGATCCTGCCTTCGGCGCCAACAGC
AACAACCCCGACTGGGACTTCAACCCCAACAAGGA
CCATTGGCCTGACGCCAACAAAGTGGGCACCACGA
CGCCAGCGCCGCGACCACCAACACCGGCGCCCACC
ATCGCGTCGCAGCCCCTGTCCCTGCGCCCAGAGGC
GTGCCGGCCAGCGGCGGGGGGCGCAGTGCACACGA
GGGGGCTGGACTTCGCCTGTGATATCTACATTTGG
GCCCCTCTGGCTGGTACTTGCGGGGTCCTGCTGCT
TTCACTCGTGATCACTCTTTACTGTAGGAGCAAGC
GGAGCAGACTGCTGCACAGCGACTACATGAACATG
ACCCCCCGGAGGCCTGGCCCCACCCGGAAGCACTA
CCAGCCCTACGCCCCTCCCAGGGATTTCGCCGCCT
ACCGGAGCAAGCGCGGTCGGAAGAAGCTGCTGTAC
ATCTTTAAGCAACCCTTCATGAGGCCTGTGCAGAC
TACTCAAGAGGAGGACGGCTGTTCATGCCGGTTCC
CAGAGGAGGAGGAAGGCGGCTGCGAACTGAGAGTG
AAGTTCAGCAGGAGCGCAGACGCCCCCGCGTACAA
GCAGGGCCAGAACCAGCTCTATAACGAGCTCAATC
TAGGACGAAGAGAGGAGTACGATGTTTTGGACAAG
AGACGTGGCCGGGACCCTGAGATGGGGGGAAAGCC
GAGAAGGAAGAACCCTCAGGAAGGCCTGTACAATG
AACTGCAGAAAGATAAGATGGCGGAGGCCTACAGT
GAGATTGGGATGAAAGGCGAGCGCCGGAGGGGCAA
GGGGCACGATGGCCTTTACCAGGGTCTCAGTACAG
CCACCAAGGACACCTACGACGCCCTTCACATGCAG
GCCCTGCCCCCTCGCGAGGGCAGGGGAAGTCTTCT
AACATGCGGGGACGTGGAGGAAAATCCCGGCCCCA
TGGTGAGCAAGGGCGAGGAGCTGTTCACCGGGGTG
GTGCCCATCCTGGTCGAGCTGGACGGCGACGTAAA
CGGCCACAAGTTCAGCGTCTCCGGCGAGGGCGAGG
GCGATGCCACCTACGGCAAGCTGACCCTGAAGTTC
ATCTGCACCACCGGCAAGCTGCCCGTGCCCTGGCC
CACCCTCGTGACCACCCTGACCTACGGCGTGCAGT
GCTTCAGCCGCTACCCCGACCACATGAAGCAGCAC
GACTTCTTCAAGTCCGCCATGCCCGAAGGCTACGT
CCAGGAGCGCACCATCTTCTTCAAGGACGACGGCA
ACTACAAGACCCGCGCCGAGGTGAAGTTCGAGGGC
GACACCCTGGTGAACCGCATCCAGCTGAAGGGCAT
CGACTTCAAGGAGGACGGCAACATCCTGGGGCACA
AGCTGGAGTACAACTACAACAGCCACAACGTCTAT
ATCATGGCCGACAAGCAGAAGAACGGCATCAAGGT
GAACTTCAAGATCCGCCACAACATCGAGGACGGCA
GCGTGCAGCTCGCCGACCACTACCAGCAGAACACC
CCCATCGGCGACGGCCCCGTGCTGCTGCCCGACAA
CCACTACCTGAGCACCCAGTCCGCCCTGAGCAAAG
ACCCCAACGAGAAGCGCGATCACATGGTCCTGCTG
GAGTTCGTGACCGCCGCCGGGATCACTCTCGGCAT
GGACGAGCTGTACAAGTAA
Human CDS signal peptide
(SEQ. ID. NO. 13)
MALPVTALLLPLALLLHAARP
```

1. A composition comprising a nucleic acid encoding SEQ ID NO: 12, such as a cell or vesicle.

2-21. (canceled)
